(19) United States
(12) Patent Application Publication
(10) Pub. No.: US 2004/0167422 A1
(43) Pub. Date: Aug. 26, 2004

(54) Breast electrode array and method of analysis for detecting and diagnosing diseases

(75) Inventors: Leslie W. Organ, Charleston, SC (US); Reza Safaee-Rad, Etobicoke (CA); Milan Graovac, Toronto (CA); Kenneth Carless Smith, Toronto (CA); Joel Steven Ironstone, Toronto (CA)

Correspondence Address: NIXON & VANDERHYE, PC, 1100 N GLEBE ROAD, 8TH FLOOR, ARLINGTON, VA, 22201-4714, US

(73) Assignee: Z-Tech (Canada) Inc., Toronto (CA)

(21) Appl. No.: 10/724,357

(22) Filed: Dec. 1, 2003

Related U.S. Application Data
(60) Provisional application No. 60/429,560, filed on Nov. 29, 2002.

(51) Int. Cl. A61B 5/053

(52) U.S. Cl. 600/547

(57) ABSTRACT

A breast electrode array and method of analysis for detecting and diagnosing diseases. In particular, the electrode array has a body, a plurality of flexible arms extending from the body, and a plurality of outer electrodes provided by the plurality of flexible arms, and a plurality of inner electrodes provided on at least one of the flexible arms and positioned partway between the body and the outer electrodes, and the outer electrodes and the inner electrodes are arranged on the arms to obtain impedance measurements between respective electrodes. Diagnostic methods based on homologous electrical difference analysis are also provided, utilizing the different topologies created by the inner and outer electrodes when taking impedance measurements.

### FIELD OF THE INVENTION

The present invention relates to an improved breast electrode array and method for detecting and diagnosing disease states in a living organism by using a plurality of electrical impedance measurements.

### BACKGROUND OF THE INVENTION

Methods for screening and diagnosing diseased states within the body are based on sensing a physical characteristic or physiological attribute of body tissue, and then distinguishing normal from abnormal states from changes in the characteristic or attribute. For example, X-ray techniques measure tissue physical density, ultrasound measures acoustic density, and thermal sensing techniques measure differences in tissue heat. Another measurable property of tissue is its electrical impedance; i.e., the resistance tissue offers to the flow of electrical current through it. Values of electrical impedance of various body tissues are well known through studies on intact humans or from excised tissue made available following therapeutic surgical procedures. In addition, it is well documented that a decrease in electrical impedance occurs in tissue as it undergoes cancerous changes. This finding is consistent over many animal species and tissue types, including, for example human breast cancers.

One technique for screening and diagnosing diseased states within the body using electrical impedance is disclosed in U.S. Pat. No. 6,122,544. In this patent data are obtained in organized patterns from two anatomically homologous body regions, one of which may be affected by disease. One subset of the data so obtained is processed and analyzed by structuring the data values as elements of an impedance matrix. The matrices can be further characterized by their eigenvalues and eigenvectors. These matrices and/or their eigenvalues and eigenvectors can be subjected to a pattern recognition process to match for known normal or disease matrix or eigenvalue and eigenvectors patterns. The matrices and/or their eigenvalues and eigenvectors derived from each homologous body region can also be compared, respectively, to each other using various analytical methods and then subject to criteria established for differentiating normal from diseased states.

Published international patent application, PCT/CA01/01788, discloses a breast electrode array for diagnosing the presence of a disease state in a living organism, wherein the electrode array comprises a flexible body, a plurality of flexible arms extending from the body, and a plurality of electrodes provided by the plurality of flexible arms, wherein the electrodes are arranged on the arms to obtain impedance measurements between respective electrodes. In one embodiment, the plurality of flexible arms are spaced around the flexible body and are provided with an electrode pair. In operation, the electrodes are selected so that the impedance data obtained will include elements of an impedance matrix, plus other impedance values that are typically obtained with tetrapolar impedance measurements. In a preferred embodiment the differences between corresponding homologous impedance measurements in the two body parts are compared in variety of ways that allow the calculation of metrics that can serve either as an indicator of the presence of disease or localize the disease to a specific breast quadrant or sector. The impedance differences are also displayed graphically, for example in a frontal plane representation of the breast by partitioning the impedance differences into pixel elements throughout the plane. These pixel plots as well can be used to define a set of metrics for cancer detection, for example by using the difference between homologous pixels of two body parts.

### SUMMARY OF THE INVENTION

This invention provides for an improved breast electrode array and method of analysis for detecting and diagnosing diseases, particularly using the improved electrode array of this invention.

In particular, an electrode array for diagnosing the presence of a disease state in a living organism is disclosed, with the electrode array comprising a body, a plurality of flexible arms extending from the body, and a plurality of outer electrodes provided by the plurality of flexible arms, and a plurality of inner electrodes provided on at least one of the flexible arms and positioned partway between the body and the outer electrodes, and wherein the outer electrodes and the inner electrodes are arranged on the arms to obtain impedance measurements between respective electrodes.

In another aspect of this invention, the electrode array comprises a body, a plurality of flexible arms extending from the body, and a plurality of outer electrodes provided by the plurality of flexible arms, the outer electrodes arranged on the arms to obtain impedance measurements between respective electrodes and with at least one of the outer electrodes spaced from the body greater than the other outer electrodes.

In particular, at least a further one of the outer electrodes is spaced from the body greater than the other outer electrodes but not as great as said at least one outer electrode. Moreover, the further outer electrode is provided on a flexible arm adjacent to a flexible arm having the at least one outer electrode.

Further, the outer electrodes are arranged in electrode pairs, and each of the plurality of arms is provided with an electrode pair. Similarly, the inner electrodes can be arranged in electrode pairs.

In a further aspect of the invention, at least one of the inner electrodes is spaced from the body greater than the other inner electrodes, and the at least one inner electrode is provided on the flexible arm having the at least one outer electrode.

The electrode array can also feature the plurality of flexible arms spaced around the body.

In a further aspect of the invention, the electrode array has the at least one outer electrode comprising a first set of electrodes having at least one electrode on each of two adjacent flexible arms. More particularly, the electrode array has the outer electrodes provide for a second set of electrodes spaced from the body greater than the other outer electrodes but not as great as the first set of electrodes, and the second set of electrodes has at least one electrode on each of two flexible arms, and the flexible arms are each adjacent to one of the flexible arms that has the first set of electrodes. A third set of electrodes are spaced from the body greater than the other outer electrodes but not as great as the second set of electrodes, and the third set of electrodes has at least one electrode provided on one flexible arm, and that flexible arm is adjacent to one of the flexible arms that has the second set of electrodes. Moreover, a fourth set of electrodes are spaced from the body greater than the other outer electrodes but not as great as the third set of electrodes, and the fourth set of electrodes has at least one electrode on each of two flexible arms, and one of the flexible arms is adjacent the flexible arm that has the third set of electrodes, and the other of said flexible arms is adjacent one of the flexible arms that has the second set of electrodes. The remaining of the other outer electrodes are equally spaced from the body not as great as the fourth set of electrodes.

The inner electrodes can be provided on at least one of the flexible arms and positioned partway between the body and the outer electrodes, and with at least one of the inner electrodes spaced from the body greater than the other inner electrodes and provided on one of the flexible arms having the first set of electrodes. Moreover, at least one of the other inner electrodes is provided on one of the flexible arms having the second set of electrodes, but not adjacent to the flexible arm having the at least one inner electrode, and at least one of the other inner electrodes is provided on the flexible arm having the third set of electrodes, and with this flexible arm not adjacent the flexible arm having both the second set of electrodes and said other inner electrodes. Further at least one of the other inner electrodes is provided on at least one other flexible arm that is not adjacent to any of the flexible arms that have the first, second, third, and fourth set of electrodes. The other inner electrodes are equally spaced from the body.

In one aspect of the invention certain of the flexible arms are of different lengths to provide for the spacing of the different sets of electrodes.

Moreover, at least one the flexible arms is transparent and is provided with a marker along the central axis of the flexible arm. The marker is a line along the central axis of the flexible arm. The flexible arm with the marker is provided with a tab at its end thereof.

In further aspect of this invention, a system for diagnosing the possibility of disease in a body part is disclosed. The system comprises an electrode array of this invention containing a plurality of outer electrodes and at least one inner electrode capable of being electrically coupled to the body part, a controller switching unit, and a multiplexing unit. The controller switching unit and multiplexing unit allow a current to flow between any two electrodes and a resultant voltage measurement to be measured between any two electrodes. In particular, the controller-switching unit and the multiplexing unit allows any one of the inner electrodes and outer electrodes to be a current injection electrode, and allows any one the inner electrodes and outer electrodes to be a voltage measurement electrode. In one aspect of the invention, the controller-switching unit and the multiplexing unit select the current injection electrodes and the voltage measurement electrodes such that a tetrapolar measurement is taken between any two pairs of inner electrodes, any two pairs of outer electrodes, and any two pairs of electrodes with one selected from the pairs of outer electrodes and one selected from the pairs of inner electrodes.

A template for positioning an electrode array on a part of a living organism to be diagnosed for the presence of a disease state is also disclosed. The template comprises an elongate body, and a mark provided over at least part of the length of the body, and wherein the elongate body has an opening therein and is provided with at least one hole spaced from the opening. In a preferred use of the template to position an electrode array of this invention to a breast, the opening is sized to fit around a nipple of the breast. In particular, the elongate body has a central axis and the mark is on the central axis. The mark can be a line along the central axis of the template. The mark extends to the other end of the elongate body. The elongate body can be transparent. The opening and the at least one hole are spaced from one another along the central axis. In a preferred aspect the at least one hole is three holes.

In one aspect, the opening is provided at one end of the elongate body, and the elongate body is of sufficient length so that when the opening is fitted around the nipple of one breast the other end of the elongate body extends to at least the nipple of the other breast.

A system for positioning an electrode array on a part of a living organism to be diagnosed for the presence of a disease state is also disclosed. In particular, the system comprises a template having an elongate body, and a mark provided over at least part of the length of the body, and wherein the elongate body has an opening therein and is provided with at least one hole spaced from the opening, and an electrode array having a body, a plurality of flexible arms extending from the body, and a marker provided along the central axis of at least one of the flexible arms.

Moreover, a method of positioning an electrode array on a part of a living organism to be diagnosed for the presence of a disease state, the electrode array positioned using a template of this invention is disclosed. The method comprises:

a. centering the opening in the template about a nipple of one breast;

b. positioning the template about the nipple until the mark on the template is at the center of the nipple of the other breast;

c. marking the living organism through the hole in the template;

d. removing the template and centering the electrode array about the nipple of the one breast; and

e. positioning the electrode array by aligning the marker provided on the at least one flexible arm to the marking on the living organism.

This invention also discloses the use of an electrode array of this invention for diagnosing the presence of a disease state in a living organism, the electrode array comprising a body, a plurality of flexible arms extending from the body, a plurality of outer electrodes provided by the plurality of flexible arms, and a plurality of inner electrodes provided on at least one of the flexible arms and positioned partway between the body and the outer electrodes, the outer electrodes and the inner electrodes are arranged on the arms to obtain impedance measurements between respective electrodes, and wherein the impedance values are arranged in a mathematical matrix and mathematical analysis is performed to diagnose for the presence of a disease state.

Further, a method of diagnosing the possibility of a disease state in one of first and second substantially similar parts of a living organism is disclosed. In particular, a use of the electrode array of this invention to obtain impedance measurements through parts of a living organism is disclosed. The method and use comprises;

a) obtaining a plurality of impedance measurements taken between a predetermined plurality of points encircling a first area of the parts;

b) obtaining a plurality of impedance measurements taken between a predetermined plurality of points encircling a second area of the parts, the second area at a different topology on the part than the first area;

c) obtaining a plurality of impedance measurements taken from a predetermined plurality of points between the first area and the second area;

d) producing at least one pixel plot from a chord plot produced by the impedance measurements taken; and

e) analyzing the pixel plot to diagnose the possibility of a disease state.

In particular, the pixel plot is a first pixel plot derived from the impedance measurements taken from the first area. The pixel plot can also be a second pixel plot derived from the impedance measurements taken from the second area. Moreover, the, pixel plot can be a third pixel plot derived from the impedance measurements taken from between the first area and the second area. The third pixel plot can be the sum of separate pixel plots that can be derived from the impedance measurements taken from between each point in the first area and the plurality of points in the second area. The separate pixel plots that make the third pixel plot are all mapped onto a common frame of reference, and can be mapped onto a common reference plane. The common frame of reference is a set of orthogonal axes intersecting a predetermined point of the part of the living organism to be diagnosed. In particular, the common reference plane is the body frontal plane.

In one aspect, the pixel plot can be a plurality of pixel plots comprising a first pixel plot derived from the impedance measurements taken from the first area, a second pixel plot derived from the impedance measurements taken from the second area, and a third pixel plot derived from the impedance measurements taken between the first area and the second area.

In a further aspect of the invention, the plurality of pixel plots further comprise an integrated plot combining the first pixel plot, the second pixel plot, and the third pixel plot.

In a preferred use of the apparatus of this invention, the part of the living organism to be diagnosed by this method is a breast. For this application, the first area is the periareolar area of the breast and the first pixel plot is a periareolar pixel plot, the second area is the base area of the breast and the second pixel plot is a base pixel plot, and the third pixel plot is a conical pixel plot derived from impedance measurements taken from a predetermined plurality of points between the periareolar area of the breast and the base area of the breast.

### BRIEF DESCRIPTION OF THE DRAWING FIGURES

For a better understanding of the present invention and to show more clearly how it may be carried into effect, reference will now be made, by way of example, to the accompanying drawings, which show a preferred embodiment of the present invention and in which:

FIG. 1 is an illustration of a four-electrode impedance measurement technique;

FIG. 2 is an illustration of a breast electrode array for the left breast in accordance with the present intention;

FIG. 3 shows a block diagram of a system for measuring a voltage in a body part, according to the teachings of the present invention;

FIGS. 4A-D shows modes of the controller switching unit of FIG. 3;

FIG. 5 shows a hybrid mode of the controller-switching unit of FIG. 3;

FIG. 6 shows electrical connections in a particular tetrapolar impedance measurement that employs the system of FIG. 3;

FIGS. 7A and 7B show the multiplexer of FIG. 3;

FIG. 8 shows a diagnostic system that includes an internal load in addition to the components of FIG. 3;

FIG. 9 shows one embodiment of the controller-switching unit;

FIG. 10 is an illustration of an alignment ruler used to define and mark the inter-nipple horizontal axis;

FIGS. 11a, 11b, 11c, and 11d, show the four conical surfaces created by connecting the four periareolar plane electrodes to the base plane electrodes;

FIG. 12 is an illustration of top plane (periareolar plane) impedance chords derived from the electrode array of FIG. 2;

FIG. 13 is an illustration of some of the base plane impedance chords derived from the electrode array of FIG. 2;

FIGS. 14a, 14b, 14c, and 14d, are illustrations of the conical plane impedance chords derived from the electrode array of FIG. 2; and

FIGS. 15a, 15b, 15c, and 15d are examples of periareolar, conical, base, and integrated pixel plots: derived from this invention.

### DESCRIPTION OF PREFERRED EMBODIMENT

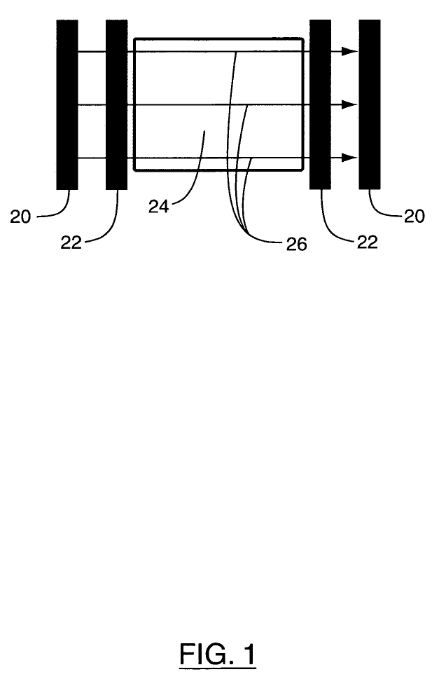

As disclosed in applicant's co-pending application Ser. No. 09/749,613, the entirety of which is incorporated herein by reference, electrical impedance is measured by using four electrodes as shown in FIG. 1. The outer pair of electrodes 20 is used for the application of current I, and the inner pair of electrodes 22 is used to measure the voltage V that is produced across a material, such as tissue 24, by the current. The arrows 26 indicate the current I flowing between electrodes 20. The impedance Z is the ratio of V to I; i.e., Z=V/I. By using separate electrodes for current injection and voltage measurement polarization effects at the voltage measurement electrodes are minimized and a more accurate measurement of impedance can be made.

Impedance consists of two components, resistance and capacitive reactance (or equivalently, the magnitude of impedance and its phase angle). Both components are measured, displayed, and analyzed in the present invention. However, for the purpose of explanation of the invention, only resistance will be used and will interchangeably be referred to as either resistance or the more general term impedance.

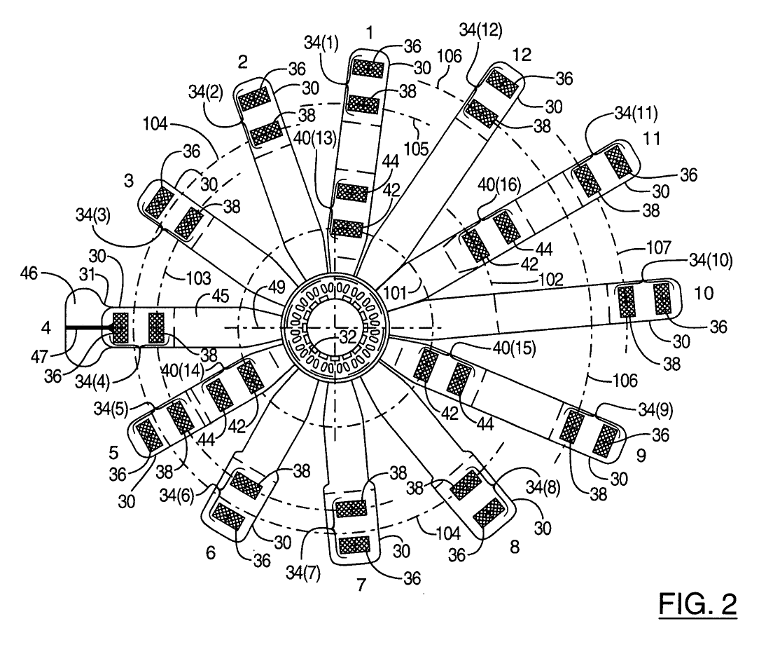

FIG. 2 discloses a preferred breast electrode array 28 of the present invention. Electrode array 28 as shown in FIG. 2 is for the left breast. An electrode array for the right breast would differ in that it is a mirror image of the electrode array illustrated in FIG. 2. Except where indicated, the following discussion for electrode array 28 would apply to either of the electrode arrays for the right breast and the left breast.

Twelve array arms 30 are shown in the electrode array 28 of FIG. 2 spaced around a body 32. Each array arm 30 is provided with at least one outer electrode, and, for the embodiment illustrated, an outer electrode pair 34, comprised of a current injection electrode 36 and voltage measurement electrode 38. The electrodes that make up the electrode pairs can be physically identical. It can be appreciated, however, that the electrodes need not be the same size or shape, nor spaced from one another as shown in FIG. 2. For example, an electrical pair could comprise one electrode as a semi-circle, and the other electrode as an interior dot to the semi-circle. Other configurations of electrodes are contemplated by this invention.

In the embodiment illustrated, twelve electrode pairs 34 are provided around the electrode array 28, with each electrode pair 34 positioned near the outer edge of each array arm 30. The electrode pairs 34 are numbered counterclockwise for the left breast electrode array, one (1) through twelve (12), with the first electrode pair one (1) positioned near the top of FIG. 2. The numbering convention for the right breast electrode array is clockwise. This allows mirror-imaged electrode pairs to be compared, which facilitates homologous comparison between breasts.

In addition an inner electrode is provided on certain of the array arms 30 of the electrode array 28. For the embodiment illustrated in FIG. 2, four inner electrode pairs 40 are provided around the electrode array 28, but positioned on the array arms 30 partway between the outer edge of the array arms and the body 32. By positioning electrode pairs 40 partway on the array arms these electrodes are placed closer to the nipple area of the breast, thus allowing better detection of cancers in the periareolar area of the breast. Again, the electrodes that make up the inner electrode pairs can be physically identical. It can be appreciated, however, that, just as for the outer electrodes, the inner electrodes need not be the same size or shape, nor spaced from one another as shown in FIG. 2. Other configurations, such as the semi-circle/interior dot arrangement described above, are contemplated by this invention.

For the embodiment illustrated, electrode pairs 40 are provided on the array arms 30 that carry the electrode pairs 34 that are numbered one (1), five (5), nine (9), and eleven (11). Electrode pairs 40 are similarly numbered counterclockwise in the left breast electrode array, thirteen (13) through sixteen (16). Again, the numbering convention for the right breast electrode array is clockwise to allow for mirror-imaged electrode pairs to be compared.

Each electrode pair 40 is comprised of a current injection electrode 42 and voltage measurement electrode 44, similar to that for electrode pairs 34. For the electrode connections illustrated, the current injection electrodes 42 and the voltage measurement electrodes 44 of the electrode pairs 40 are in an opposite orientation to the current injection electrodes 36 and voltage measurement electrodes 38 of electrode pairs 34. These orientations of the electrodes maintain the required positioning of I, V, V, I (as shown in FIG. 1) for tetrapolar measurement between outer electrode pairs 34 and inner electrode pairs 40.

It is to be noted, however, that the terms “current injection” and “voltage measurement” refer to the use of any four electrodes used for tetrapolar impedance measurement, with the two electrodes between which current is injected being called current injection electrodes, and the two electrodes across which voltage is measured being called voltage measurement electrodes. In particular, the present invention has the capability of interchanging which electrodes are used for current injection and voltage measurement. This allows, for example, impedance measurements to be taken between any two of electrode pairs 40, numbered thirteen (13), fourteen (14), fifteen (15) and sixteen (16), in FIG. 2. For purposes of these measurements, electrodes 44 are used for current injection and electrodes 42 are used for voltage measurement. This allows the arrangement of I,V,V,I, shown in FIG. 1 to be maintained.

A diagnostic system capable of interchanging which electrodes are used for current injection and voltage measurement will now be described. Moreover, the diagnostic system is capable of tetrapolar measurements, as described above, and also of bipolar measurements where a single electrode is used for both current injection and voltage measurement. For example, current is injected between two electrodes and voltage is measured between the same two electrodes.

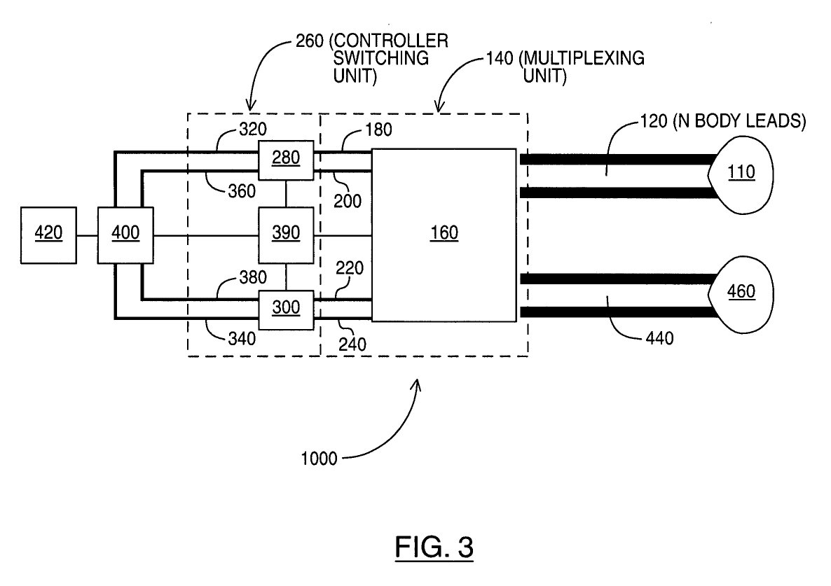

FIG. 3 shows a diagnostic system 1000 for measuring a voltage in a body part 110, such as a human breast. The system 1000 includes N body leads 120. In what follows, the N body leads 120 are ordered from 1 to N for reference. The system 1000 also includes a multiplexing unit 140 having a multiplexer 160, a first MX lead 180, a second MX lead 200, a third MX lead 220 and a fourth MX lead 240.

The system 1000 further includes a controller switching unit 260 having a first switch 280 connected to the multiplexer 160 by the first MX lead 180 and the second MX lead 200, a second switch 300 connected to the multiplexer 160 by the third MX lead 220 and the fourth MX lead 240, a current input lead 320 connected to the first switch 280, a current output lead 340 connected to the second switch 300, a first voltage lead 360 connected to the first switch 280, and a second voltage lead 380 connected to the second switch 300. The controller switching unit 260 also includes a controller 390. The system 1000 further includes an impedance module 400 and a diagnosis module 420.

Also shown in FIG. 3 is an optional second set of leads 440 that can be used when making measurements on a second homologous body part 460. The description below is directed mainly to an impedance measurement on the one body part 110 with the set of N leads 120, but it should be understood that the discussion could be analogously expanded to include an impedance measurement on the second homologous body part 460 with the second set of leads 440. Thus, the principles of the present invention can be applied to diagnosis of disease by making electrical measurements on a single body part, or by making measurements on a homologous pair of body parts When making measurements on only a single body part, the results can be compared to standard results obtained from population studies, for example, to diagnose disease. When using a homologous pair of body parts, the results of one body part can be compared to the results of the homologous body part of the same patient, as described in U.S. Pat. No. 6,122,544.

The N body leads 120 electrically connect the multiplexing unit 140 to the body part 110. Each of the N body leads 120 includes a wire capable of carrying a current and an electrode to attach to the body part 110. A current conducting gel can act as an interface between the electrode and the skin covering the body part 110.

The multiplexing unit 140 and the controller switching unit 260 allow a current to flow through the body part 110 between any two body leads, n₁, and n₂, of the N body leads 120, and a resultant voltage to be measured between any two body leads, n₃ and n₄, of the N body leads 120, where n₁≠n₂ and n₃≠n₄, but where n₁, n₂, n₃ and n₄, need not otherwise be distinct. Thus, n₁, n₂, n₃, and n₄ are numbers belonging to the set {1,2, . . . ,N} that identify body leads. For example, if n₁=7, then n₁ denotes the seventh body lead from among the N body leads 120 used to inject current into the body part 110.

The impedance module 400 generates current that is injected into the current input lead 320 and then delivered to the body part. The current output lead 340 receives the current from the body part. When the current is traveling through the body part, the first voltage lead 360 and the second voltage lead 380 are used to measure the resultant voltage between these leads 360 and 380. The impedance module 400 uses this voltage, together with the known current injected into the current input lead 320, to calculate corresponding impedance, which may then be used by the diagnosis module 420 to diagnose disease.

In one embodiment, N is even and the multiplexer 160 can electrically connect the first MX lead 180 and the fourth MX lead 240 to a first set of N/2 of the N leads, and the second MX lead 200 and the third MX lead 220 to a second set of the other N/2 leads. In a conventional system, the first set of N/2 leads are exclusively used to inject current into and receive current from the body part. The second set of N/2 leads are then exclusively used to measure resultant voltages in tetrapolar measurements. This configuration limits the number of impedances that can be measured.

In the system 1000, however, the second set of N/2 leads can also be used to inject and receive current, and the first set can be used to measure resultant voltages. Thus, the system 1000 can furnish a greater number of impedances. Moreover, as detailed below, the system can make both tetrapolar and bipolar measurements. The added benefits arise from the functionality of the controller switching unit 260. By using the controller switching unit 260, the system 1000 can force current to flow through the body part 110 between any two body leads, n₁ and n₂, of the N body leads 120, and a resultant voltage to be measured between any two body leads, n₃ and n₄ of the N body leads 120, where n₁≠n₂ and n₃ ≠n₄.

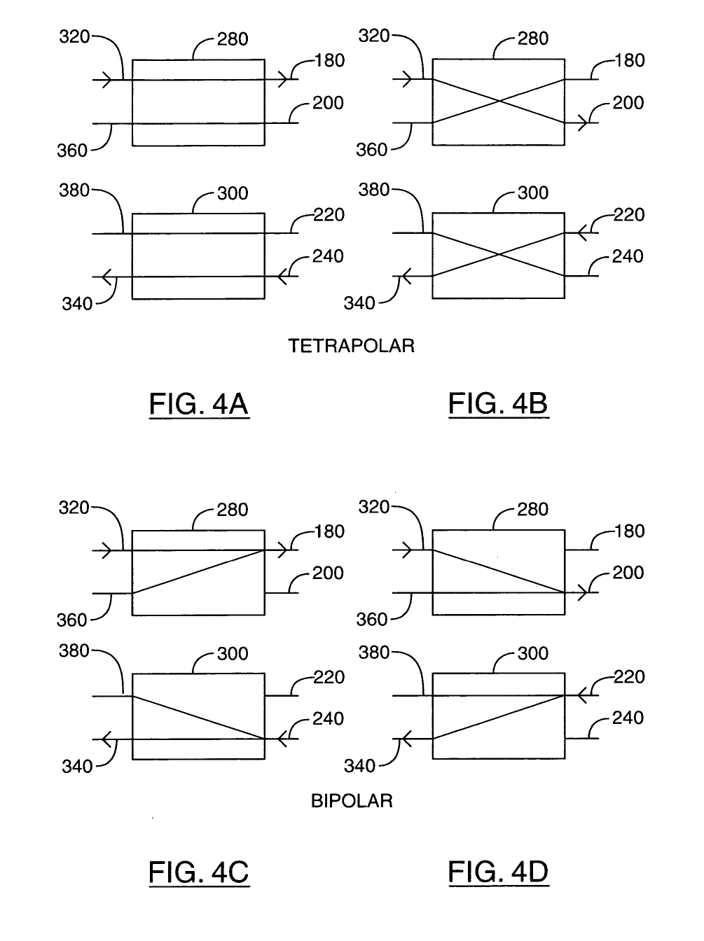

FIGS. 4A-D show several states of the switches 280 and 300 resulting in different modes of the controller switching unit 260 of the system of FIG. 3. These states of the switches 280 and 300 are controlled by the controller 390. In FIG. 4A, current is injected into the first MX lead 180 and received by the fourth MX lead 240. While this current travels through the body part 110, a resultant voltage is measured between the second MX lead 200 and the third MX lead 220. This measurement is tetrapolar because current is forced to flow between two leads and the resultant voltage is measured between two other leads.

In FIG. 4B, current is injected into the second MX lead 200 and received by the third MX lead 220. The resultant voltage is measured between the first MX lead 180 and the fourth MX lead 240. This measurement is also tetrapolar.

In FIGS. 4A and 4B, the first switch 280 and the second switch 300 are both in tetrapolar states since, for each of the switches 280 and 300, two distinct MX leads are involved in the impedance measurement. When both switch states are tetrapolar, the controller switching unit 260 is said to be in a tetrapolar mode. Thus, FIGS. 4A and 4B correspond to tetrapolar modes.

In a tetrapolar mode, the current input lead 320 is electrically connected to exactly one of the first MX lead 180 and the second MX lead 200 and the first voltage lead 360 is electrically connected to the other one of the first MX lead 180 and the second MX lead 200; likewise, the current output lead 340 is electrically connected to exactly one of the third MX lead 220 and the fourth MX lead 240 and the second voltage lead 380 is connected to the other one of the third MX lead 220 and the fourth MX lead 240.

The two tetrapolar modes shown in FIGS. 4A and 4B do not exhaust all the tetrapolar modes. For example, when the first switch 280 state is the same as the state shown in FIG. 4A and the second switch 300 state is the same as the state shown in FIG. 4B, the controller switching unit 260 is also in a tetrapolar mode. Generally, the controller switching unit 260 is in a tetrapolar mode when n₁,n₂,n₃ and n₄ are distinct, where n₁ and n₂ are leads from among the N leads 120 used to inject current into and receive current from the body part 110, and n₃ and n₄ are leads used to measure the resultant voltage.

In FIG. 4C, current is injected into the first MX lead 180 and received by the fourth MX lead 240. While this current travels through the body part 110, a resultant voltage is measured between the first MX lead 180 and the fourth MX lead 240. The second and third MX leads 200 and 220 are electrically unconnected to any of the N body leads 120 during this measurement. This measurement is bipolar because the pair of electrodes used for measuring a voltage is also used for current flow.

In FIG. 4D, current is injected into the second MX lead 200 and received by the third MX lead 220. The resultant voltage is measured between the same two leads 200 and 220. The first and fourth MX leads 180 and 240 are electrically unconnected during this measurement. This measurement is also bipolar.

In FIGS. 4C and 4D, the first switch 280 and the second switch 300 are both in bipolar, states since, for each of the switches 280 and 300, only one MX lead is involved in the impedance measurement. When both switch states are bipolar, the controller switching unit 260 is said to be in a bipolar mode. Thus, FIGS. 4C and 4D correspond to bipolar modes.

In a bipolar mode, the current input lead 320 and the first voltage lead 360 are electrically connected to each other and to exactly one of the first MX lead 180 and the second MX lead 200, and the current output lead 340 and the second voltage lead 380 are electrically connected to each other and to exactly one of the third MX lead 220 and the fourth MX lead 240.

The two modes shown in FIGS. 4C and 4D do not exhaust all bipolar modes. For example, when the first switch 280 state is the same as the state shown in FIG. 4C and the second switch 300 state is the same as the state shown in FIG. 4D, the controller switching unit 260 is also in a bipolar mode. More generally, the controller switching unit 260 is in a bipolar mode when n, n₃ or n₄, and n₂ n₃ or n₄, where n₁ and n₂ are leads from among the N leads 120 used to inject and receive current, and n₃ and n₄ are leads used to measure the resultant voltage.

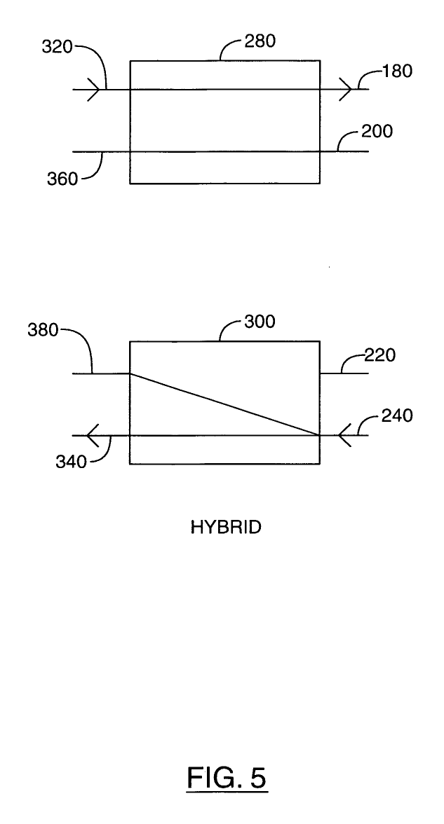

In addition to the tetrapolar and bipolar modes shown in FIGS. 4A-D, there are also hybrid modes. FIG. 5 shows a hybrid mode of the controller switching unit 260 of FIG. 3. Here, the first switch 280 is in a tetrapolar state and the second switch 300 is in a bipolar state. In a hybrid mode, n₁≠n₃ and n₂=n₄, or n₁≠n₄ and n₂=n₃, where again n₁ and n₂ are used for current flow and n₃ and n₄ are used for voltage measurement.

In FIG. 5, the lead n₁ is electrically connected to the first MX lead 180 or to the fourth MX lead 240 via the multiplexer 160. The lead n₂ is connected to whichever of first MX lead 180 and the fourth MX lead 240 is not connected to the lead n₁. The lead n₃ is connected to the second MX lead 200 or the fourth MX lead 240, and the lead n₄ is connected to whichever of the second MX lead 200 and the fourth MX lead 240 is not connected to the n₃ lead. The third MX lead 220 is electrically unconnected during this hybrid measurement.

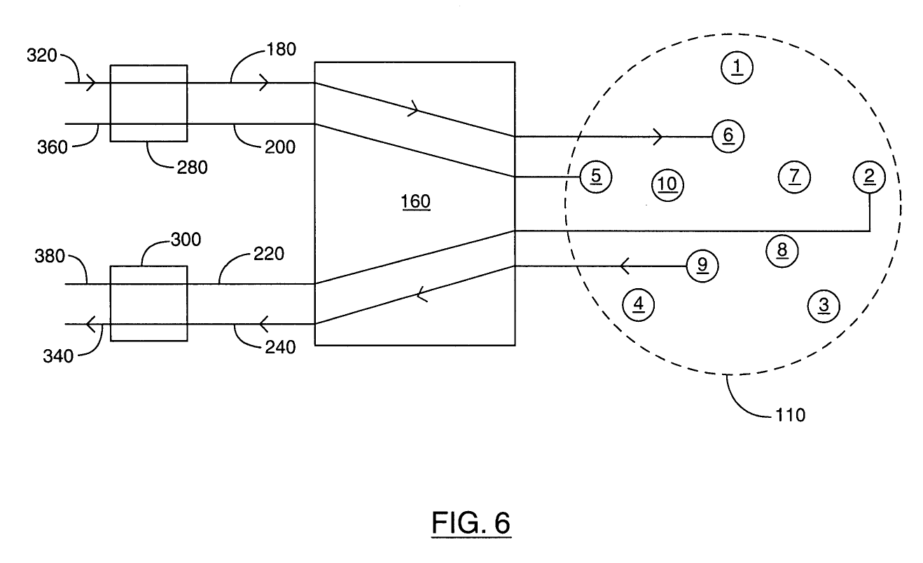

FIG. 6 shows electrical connections in a particular tetrapolar impedance measurement that employs the system 1000 of FIG. 3. For simplicity, the system 1000 has only N=10 leads, and the controller 390, the impedance module 400 and the diagnosis module 420 are not shown. In a different embodiment, N=32. Also not shown in FIG. 6 is the second set of leads 440. The ten electrodes 10f the ten leads are shown: the first set of N/2=five electrodes 1-5 lie on the outside perimeter and the other set of five electrodes 6-10 lie on the inner perimeter. It can be appreciated that the model of FIG. 6, for purposes of this discussion, can be applied to the outer electrode pairs—34 numbered one (1) through twelve (12)—and the inner electrode pairs 40—numbered thirteen (13) through sixteen (16)—of the electrode array 28 illustrated in FIG. 2. Applications to other electrode arrays of differing shapes and having different numbers of electrodes is also intended.

From FIG. 6, all the electrodes 1-5 of the first set can be electrically connected to the first and fourth MX leads 180 and 240, and all the electrodes 6-10 of the second set can be connected to the second and third MX leads 200 and 220 via the multiplexer 160. In the example of FIG. 6, the connections shown are for one tetrapolar measurement in which n₁=6, n₂=9, n₃=2 and n₄=5, where electrode 6 is used to inject current into the body part 110 and electrode 9 is used to receive the current. The electrodes 2 and 5 are used to measure the resultant voltage. Although all electrodes of the ten leads are shown in FIG. 6, only the four wires of the electrically active leads appear for purposes of illustration.

In particular, current is generated by the impedance module 400 and sent to the current input lead 320. From there, the current travels to the first MX lead 180 via the first switch 280 and from there to the electrode 6 via the multiplexer 160. The current next travels through the body part 110 (such as, for example, a breast) to the electrode 9 and then through the multiplexer 160 to the fourth MX lead 240. The current then flows to the current output lead 340 via the second switch 300 and then back to the impedance module 400. The resultant voltage is measured between the first and second voltage leads 360 and 380, which corresponds to the voltage between the electrodes 2 and 5. The first voltage lead 360 is connected to the electrode 2 via the first switch 280 and the multiplexer 160, and the second voltage lead 380 is electrically connected to the electrode 5 via the second switch 300 and the multiplexer 160. The controller 390 controls the states of the switches 280 and 300 and the multiplexing states in the multiplexer 160 that determine through which leads current flows and which leads are used to measure voltage.

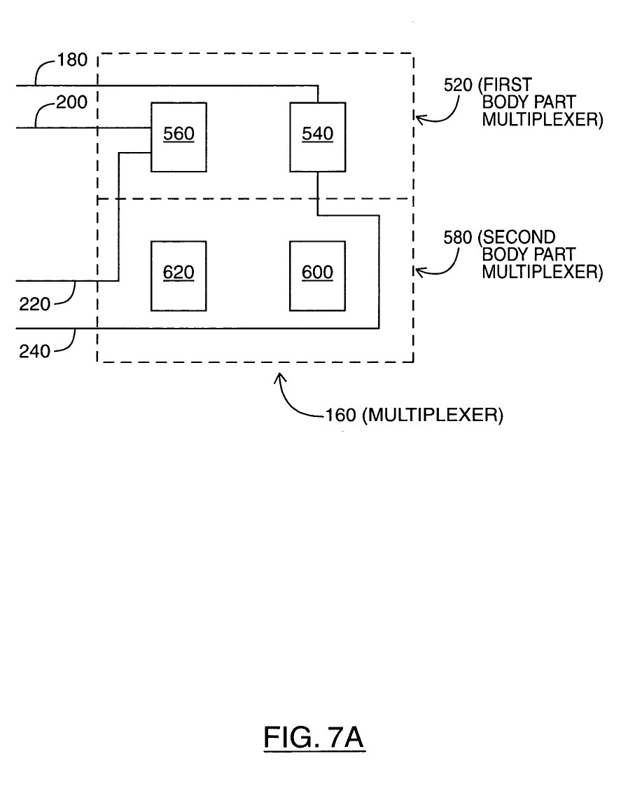

FIG. 7A shows the multiplexer 160 of FIG. 3 in an embodiment in which a body part is being compared to a homologous body part. The multiplexer 160 includes a first body part multiplexer 520 that includes a first body part A multiplexer unit 540 and a first body part B multiplexer unit 560. The multiplexer 160 also includes a second body part multiplexer 580 that includes a second body part A multiplexer unit 600 and a second body part B multiplexer unit 620. The first body part A multiplexer unit 540 is connected to the first MX lead 180 and the fourth MX lead 240. The first body part B multiplexer unit 560 is connected to the second MX lead 200 and the third MX lead 220. Although not shown in the interest of clarity, the second body part A multiplexer unit 600 is also connected to the first MX lead 180 and the fourth MX lead 240, and the second body part B multiplexer unit 620 is also connected to the second MX lead 200 and the third MX lead 220.

The first body part multiplexer 520 is used for multiplexing electrical signals to the first body part of the homologous pair. In particular, the first body part A multiplexer unit 540 and B multiplexer unit 560 are both capable of multiplexing current and voltage signals to and from the N leads 120. Likewise, the second body part multiplexer 580 is used for multiplexing electrical signals to the homologous body part. In particular, the second body part A multiplexer unit 600 and B multiplexer unit 620 are both capable of multiplexing current and voltage signals to and from the N leads 120, as described below.

FIG. 7B shows the first body part A multiplexer unit 540 of FIG. 7A. The multiplexer unit 540 includes four one-to-N/4 multiplexers 640, 660, 680 and 700. These, for example, can be model number MAX4051ACPE manufactured by MAXIM™. The N/4 multiplexer current leads 720 connect the multiplexer 640 to the multiplexer 680, and N/4 multiplexer current leads 740 connect the multiplexers 660 and 700. In turn, the leads 720 and 740 are connected to the first N/2 of the N leads 120. The multiplexers 640, 660, 680 and 700 each have a configurable one bit “inhibit state” and log₂(N/4) bit “control state.” The inhibit state can be either off (0) or on (1) and determines whether current can flow through the respective multiplexer 640, 660, 680 or 700. The control state determines through which one of the leads 720, 740 current flows. If N=32, then four bits are required for each active multiplexer (by “active” is meant that the inhibit state is off) and to specify a state, one for the inhibit state and three for the control state. For example, if the inhibit state of the multiplexer 640 is 1 (on) and the state of the multiplexer 660 is (0,0,0,1), where the first bit is for the inhibit state, and the last three bits identify which lead of multiplexer 660 is being activated, then current destined for the breast is directed to the tenth lead, provided the states of the switches 280 and 300 connect the current input lead 320 to the first MX lead 180, as previously described. If the states of the switches 280 and 300 do not connect the current input lead 320 to the first MX lead 180, but do connect the first voltage lead 360 to the first Mx lead 180, then this lead 180, when the multiplexer 660 is in the state (0,0,0.1), measures the resultant voltage with the tenth lead.

A similar binary code for the multiplexers 680 and 700 dictates through which one of the first 16 electrodes of the 32 leads 120 current is received from the breast, provided the states of the switches 280 and 300 connect the current output lead 340 to the fourth MX lead 240. If the fourth MX lead 240 is not connected to the current output lead 340, but is connected to the second voltage lead 220, then the fourth MX lead 240 is used for measuring the resultant voltage, provided the inhibit state of the multiplexer 680 or the multiplexer 700 is off

The B multiplexer unit 560 is similar to the A multiplexer unit 540 in that it has four one-to-N/4 multiplexers analogous to 640, 660, 680 and 700. However, the one-to-N/4 multiplexers are capable of connecting with the second and third MX leads 200 and 220, instead of the first and fourth MX leads 180 and 240. Here, the inhibit and control states determine which electrode from among the other N/2 electrodes is used to deliver current or measure voltage.

Thus, by setting inhibit and control states, in coordination with the states of the switches 280 and 300, it is possible to direct current between any pair of the N leads 120 and to make a measurement of the resultant voltage between any pair of the N leads 120.

The inhibit and control states are set by the controller 390 with a shift-register and/or a computer. A direct digital stream can be sent to the shift register for this purpose.

The function of the second body part multiplexer 580 is analogous to that of the first body part multiplexer 520 and therefore need not be described further.

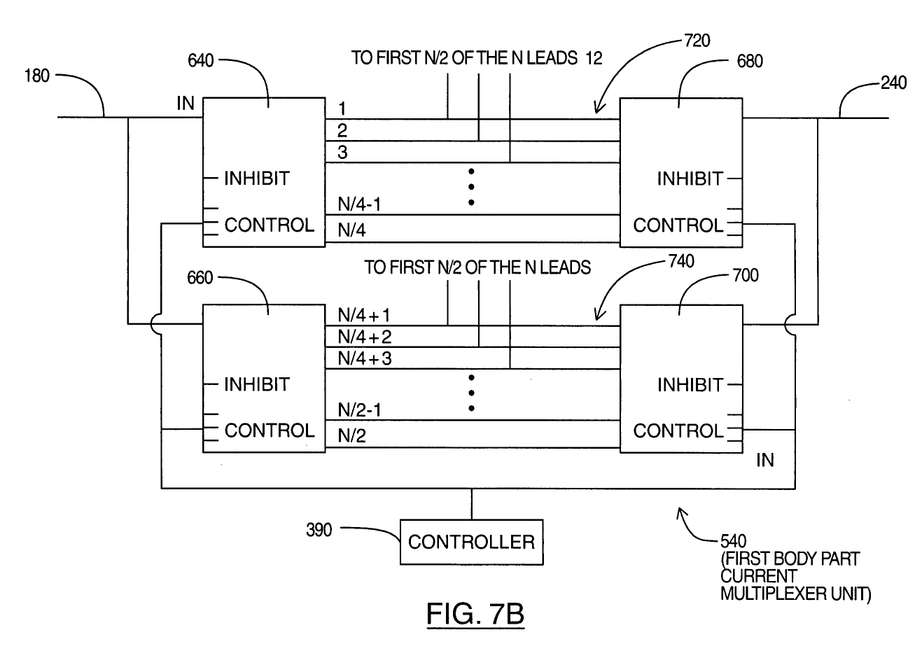

FIG. 8 shows a diagnostic system 820 that includes an internal load 840 in addition to the components described above in relation to FIG. 3. The internal load 840 is electrically connected to the first MX lead 180, the second MX lead 200, the third MX lead 220 and the fourth MX lead 240. The internal load 840 is used for at least one of internal testing of the system 820 and varying the measurment range of the system 820.

Using the first switch 280 and the second switch 300, the internal load 840 can be connected to the impedance module 400 in a tetrapolar mode or in a bipolar mode. The internal load 840 has a known impedance and therefore can be used to test the diagnostic system 820.

Additionally, the internal load 840 can be used to change the measurement range of the system 820. By attaching this internal load 840 in parallel with any load, such as the body part 110, the system 820 is capable of measuring larger impedances than would otherwise be possible. If the resistance of the internal load 840 is Rᵢₙₜ and is in parallel, the measured resistance R is given by

R=(1/R_{load}+1/Rᵢₙₜ)⁻¹

where R_{load} is the resistance of the load. Consequently, the measured resistance is reduced from the value without the internal load, thereby increasing the measurement range of the system 840.

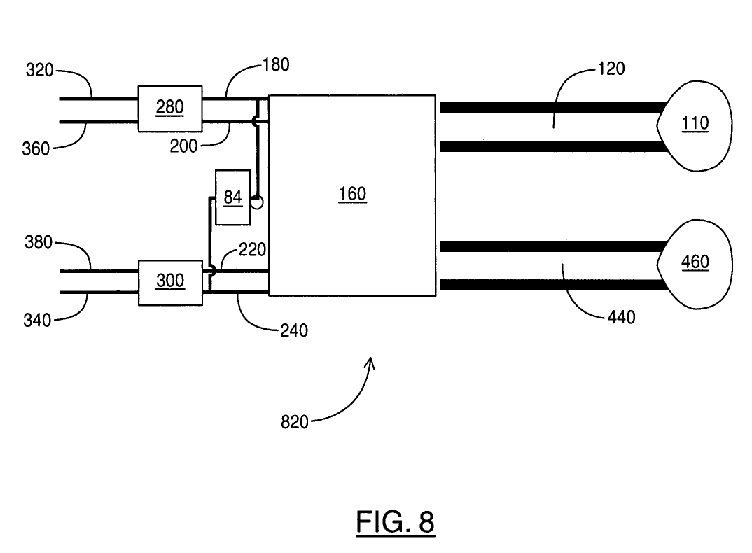

The switches 280 and 300 allow current to flow between various pairs of electrodes on a body part, and resultant voltage to be measured between various pairs of electrodes, as described above with reference to FIGS. 3-8. In FIG. 9, another embodiment of the controller switching unit is shown that can be used to achieve the states of FIGS. 4A-D using a different electrical circuit topology. The controller switching unit 900 of FIG. 9 includes a first switch 920 and a second switch 940. The current input lead 320, the current output lead 340, the first voltage lead 360 and the second voltage lead 380 split to connect to both the first and second switches 920 and 940.

The switches 920 and 940 can be turned on or off and can be used to make tetrapolar and bipolar measurements. With only one of the switches 920 and 940 on, a tetrapolar measurement can be made. With both switches 920 and 940 on, a bipolar measurement can be made. For example, when the first switch 920 is on, and the second switch is off, the resultant functionality corresponds to that of FIG. 4A, albeit achieved with a different circuit topology. In this example, current flows from the impedance module 400 along the current input lead 320, through the first switch 920, and then to the first MX lead 180. From there, the current proceeds to the multiplexer 160. Current is received from the multiplexer 160 along the fourth MX lead, and delivered to the current output lead 340 via the first switch 920. The resultant voltage is measured between the second and third MX leads 200 and 220 with the use of the first and second voltage leads 360 and 380.

In another example, when the first switch 920 is off, and the second switch 940 is on, the resultant functionality corresponds to that of FIG. 4B. Here, current from the impedance module 400 travels along the current input lead 320, across the second switch 940, then jumps to the second MX lead 200. Current is received along the third MX lead 220, from where it jumps to the current output lead 340 via the second switch 940. The voltage is measured between the first and fourth MX leads 180 and 240 with the use of the first and second voltage leads 360 and 380.

In yet another example, the first and second switches 920 and 940 are both on, which corresponds to FIGS. 4C or 4D. Precisely to which of thes two figures this example corresponds is determined by the inhibit states of the multiplexer 160. For example, if the inhibit states of both of the one-to-N/4 multiplexers 640 and 660 are on, then bipolar measurements are performed with the second set of N/2 electrodes.

The controller switching unit 900 also includes an internal load switch 1080 that is connected to the internal load 840. The controller switching unit 900 and the internal load 840 are used to test the system and to increase the measurement range, as described above.

Referring again to FIG. 2, certain of array arms 30 can be of different lengths. This allows certain electrode pairs 34 and 40 to be spaced from body 32 at different positions along array arms 30, as will hereinafter become apparent. For the embodiment illustrated in FIG. 2, the array arms 30 having electrode pairs three (3), four (4), five (5), six (6), and seven (7) are of the same length. The array arms 30 having electrode pairs two (2) and eight (8) are of the same length, but slightly longer than the arms having electrode pairs three (3) through seven (7), inclusive. Similarly, the array arm 30 having electrode pair one (1) is again slightly longer. Then array arms 30 having electrode pairs nine (9) and twelve (12) are of the same size, but again still longer. Finally, array arms 30 having electrode pairs ten (10) and eleven (11) are the same size and are the longest. In all instances, electrode pairs 34 are positioned at the same location on array arms 30 near the outer edge. As a consequence, electrode pairs ten (10) and eleven (11) are spaced furthest from body 32, as illustrated in FIG. 2, followed by electrode pairs nine (9) and twelve (12), then by electrode pair one (1), then electrode pairs two (2) and eight (8), and then finally, electrode pairs three (3), four (4), five (5), six (6), and seven (7), as described above.

In addition, certain inner electrode pairs 40 can be spaced from body 32 at different positions along array arms 30. For the embodiment illustrated in FIG. 2, electrode pairs thirteen (13), fourteen (14), and fifteen (15) are spaced the same length from body 32 along their respective array arms. Electrode pair sixteen (16) is spaced from body 32 along its respective array arm further than the other electrode pairs 40.

It can therefore be appreciated that the resultant array shape illustrated in FIG. 2 is non-circular, so that when the breast electrode array 28 is applied to the left breast, oriented such that array arm 30 containing electrode pair four (4), specifically denoted here as array arm 45, is in alignment with the horizontal chest axis (as will hereinafter be explained), the greater extension of certain of the array arms will be toward the upper outer quadrant of the breast. It is also noted that left and right breast electrode arrays 30 are mirror images of one another to maintain the preferred extension to the upper outer quadrants of both breasts. In particular, by having the array arms containing electrode pair numbers ten (10), eleven (11) and twelve (12) the longest, these electrode pairs cover more fully breast tissue in the upper outer quadrant, the region where almost one-half of breast cancers occur.

It can be appreciated that different array sizes can be produced to accommodate different breast sizes. For different sizes of electrode arrays as illustrated in FIG. 2 for use with different sizes of breasts, for example, small, medium, and large, it has been found that the electrode pairs can cover more fully breast tissue in the upper quadrant if the following relationship is used. First, the position of the innermost electrodes 42 of inner electrode pairs 40, numbered thirteen (13), fourteen (14), and fifteen (15), from the center of the body 32 is identified by the concentric dotted circle 101. Setting the distance of concentric circle 101 from the center of the body 32 to one (1), then the relative distances of the others electrodes can be found as follow: for electrode 42 of electrode pair sixteen (16), identified by concentric dotted circle 102, at 1.65; for electrodes 38 of electrode pairs three (3), four (4), five (5), six (6), and seven (7), identified by concentric circle 103, at 1.83; for electrodes 38 of electrode pairs two (2) and eight (8), identified by concentric circle 104, at 2.06; for electrode 38 of electrode pair one (1), identified by concentric circle 105, at 2.24; for electrodes 38 of electrode pairs nine (9) and twelve (12), identified by concentric circle 106, at 2.60; and for electrodes 38 of electrode pairs ten (10) and eleven (11), identified by concentric circle 107, at 2.98.

Although the electrode array of FIG. 2 shows array arms of different lengths it can be appreciated that other lengths and configurations are possible. For example, all the array arms could be of the same length. Here the electrode pairs could be positioned at different locations on the respective array arms to achieve different spacing of the electrode pairs from the body 32. It can be appreciated that other lengths and configurations are possible to cover the upper outer quadrant of the breast, or any other region of the breast to be targeted, or, more generally, of a body part to be diagnosed.

Array arm 45—numbered four (4) in FIG. 2—differs from other array arms 30 by the presence of a tab 46 at its end 31. Tab 46 has a tab line 47 printed along the central axis 49 of the arm 45. For the electrode array 28 illustrated in FIG. 2, at least array arm 45 is transparent, and preferably all the array arms are transparent. This allows the subject's skin to be seen beneath tab line 47.

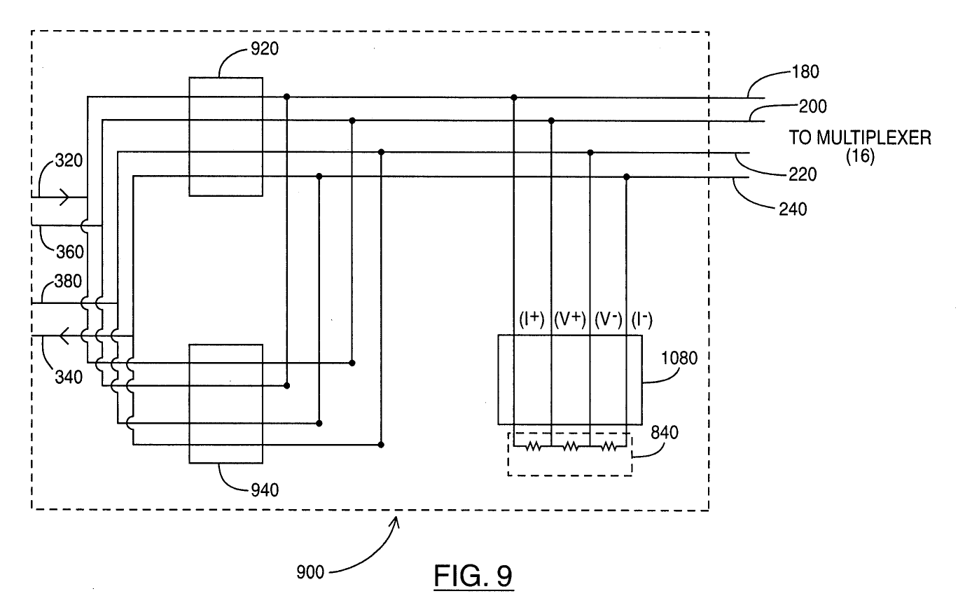

Prior to application of the breast electrode arrays, a template is used to position the electrode arrays. As illustrated in FIG. 10, the template is an alignment ruler 50 that is positioned so that circular opening 51 is centered about one nipple, then the alignment ruler 50 is rotated so that guideline 52 crosses the center of the opposite nipple to bring the guide line into the inter-nipple (horizontal) axis. Depending on the size of the breast electrode array to be used—for example, small (S), medium (M), or large (L)—a marker pen is inserted through the appropriate marker hole 53—which can be labeled small (S), medium (M), large (L)—to make an alignment mark on the subject in the inter-nipple axis. Alignment ruler 50 is then applied to the other nipple, centering circular opening 51 about it then rotating the ruler to bring guideline 52 over the center of the first nipple. A second mark is made on the subject through the same marker hole as used at the other breast. The result: two alignment marks on the skin at the medial aspect of each breast in the inter-nipple line.

Identical positioning of left and right breast electrode arrays is assured by centering the body 32 of the array over the nipple, then with the nipple as the pivot point for rotation, bringing tab line 47 over the previously placed skin alignment mark. This process is facilitated by the presence of tab 46 because (1) it allows the operator to see tab line 47 while still grasping the end of array arm 45, and (2) performing the rotation of the array at the end of the arm rather than at the body 32 reduces adjustment overshoot during the alignment process.

With the exception of the above differences, the construction of electrode array 28 is as described in applicant's co-pending application Ser. No. 09/749,613, which is incorporated herein by reference.

One technique for screening and diagnosing diseased states within the body using electrical impedance is disclosed in U.S. Pat. No. 6,122,544, and in co-pending application Ser. No. 09/749,613, which are incorporated herein by reference. In U.S. Pat. No. 6,122,544 data are obtained in organized patterns from two anatomically homologous body regions, one of which may be affected by disease. One subset of the data so obtained is processed and analyzed by structuring the data values as elements of an impedance matrix. The matrices can be further characterized by their eigenvalues and eigenvectors. These matrices and/or their eigenvalues and eigenvectors can be subjected to a pattern recognition process to match for known normal or disease matrix or eigenvalue and eigenvectors patterns. The matrices and/or their eigenvalues and eigenvectors derived from each homologous body region can also be compared, respectively, to each other using various analytical methods and then subject to criteria established for differentiating normal from diseased states.

In co-pending application, serial no. 091749,613, electrodes are selected so that the impedance data obtained can be considered to represent elements of an impedance matrix. Then two matrix differences are calculated to obtain a diagnostic metric from each. In one, the absolute difference between homologous right and left matrices, on an element-by-element basis, is calculated; in the second, the same procedure is followed except relative matrix element difference is calculated. These techniques as disclosed above can be applied utilizing the electrode array of the present invention, for example, electrode array 28 illustrated in FIG. 2.

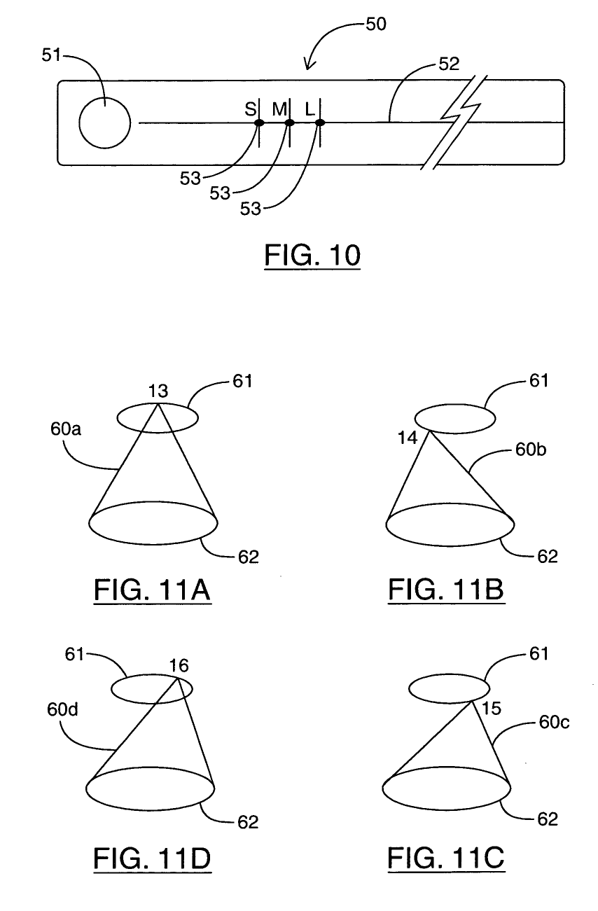

Breast electrode array 28, as constructed, is flat, but the arms are flexible, so that when applied to the breast the array shape becomes approximately a section of a sphere. It can be appreciated therefore that by placing certain of the electrodes pairs 40 at some intermediate location along array arm 30 that they will be at a different topology from electrode pairs 34. For the electrode array 28 illustrated in FIG. 2 and suitable for use in taking impedance measurements of the breast, the twelve electrode pairs 34 are closest to the chest wall, and are called base plane electrodes. These electrodes are situated in the frontal body plane. The four electrode pairs 40, whereas not precisely in the same plane, are, for the electrode array 28 illustrated in FIG. 2, close to the nipple region of the breast, and are called periareolar plane electrodes. This plane is coplanar with the base plane. Impedance measurements can be taken between each periareolar electrode pair and each of the twelve base plane electrode pairs. This will describe four conical surfaces as shown in FIGS. 11a, 11b, 11c, and 11d, with one of the periareolar plane electrodes at the apex of each cone. FIGS. 11a, 11b, 11c, and 11d show the geometrical models for these cones—60a, 60b, 60c, and 60d, respectively. The four electrode pairs 40—namely, electrode pairs thirteen (13), fourteen (14), fifteen (15), and sixteen (16)—describe the periareolar plane 61. The twelve electrode pairs 34—namely, electrode pairs one (1) through twelve (12)—describe the base plane 62. The formation of six electrode planes, as will be described below, namely, a base plane, four conical planes, and a periareolar plane, will increase the 3-dimensional sensitivity and localization accuracy of the described technology, as will hereinafter become apparent.

It is known that electrical current does not flow in a single or in a straight path through tissue. However, for purposes of the following analyses, it will be assumed it does. Because many of these analyses are based on comparison of homologous (mirror image) small areas (pixels) in each breast, the potential inaccuracies that could result from the above assumption will tend to be negligible. Therefore, current flow, and subsequent impedance measurement between electrode pairs can be represented as straight lines, or chords, connecting the two pairs.

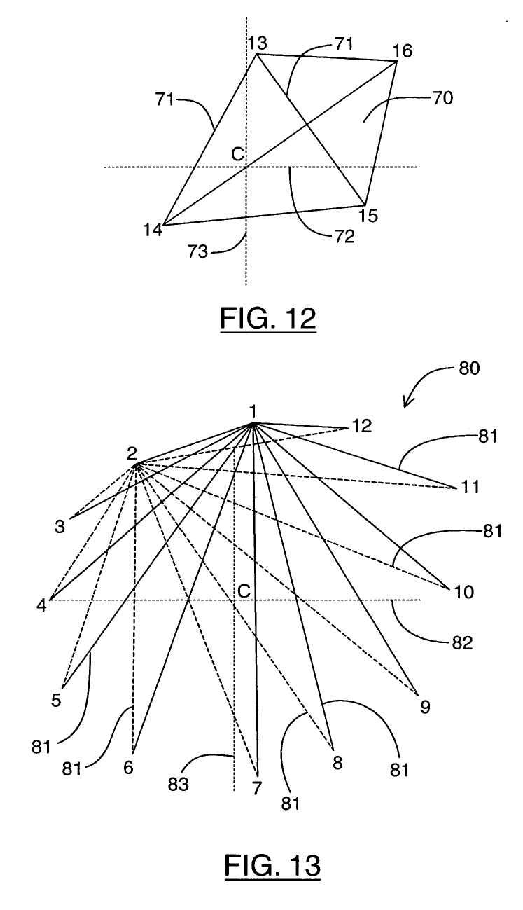

FIG. 12 shows the periareolar plane 70 with impedance chords 71 connecting the electrode pairs numbered thirteen (13) through sixteen (16). There are a total of six impedance chords 71 in this plane for the four inner electrode pairs of the electrode array 28, as illustrated in FIG. 2. Lines 72 and 73 are orthogonal axes intersecting at point C, which, for the preferred use of the electrode array 28, represents the projected position of the nipple on this plane. Lines 72 and 73 are superimposed on the plane 70 to provide a common reference between FIG. 12 and FIGS. 13 and 14, as will hereinafter become apparent.

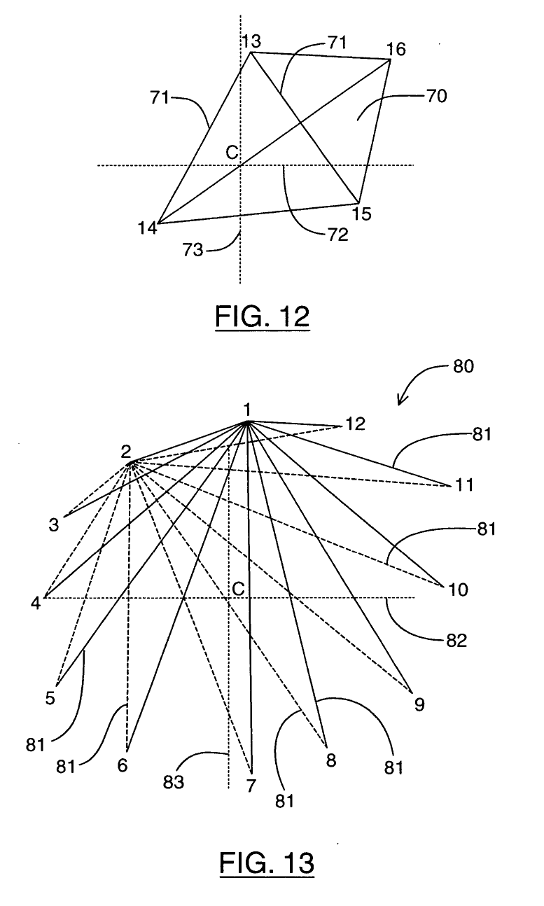

FIG. 13 shows the base plane 80 with impedance chords connecting the electrode pairs numbered one (1) through twelve (12). There are 66 impedance chords 81 in this plane (frontal body plane). Shown in FIG. 13, for illustrative purposes, are the (solid line) impedance chords emanating from electrode pair one (1) and the (dashed line) impedance chords emanating from electrode pair (2). Lines 82 and 83 are orthogonal axes intersecting at point C, which represents the position of the nipple on this plane.

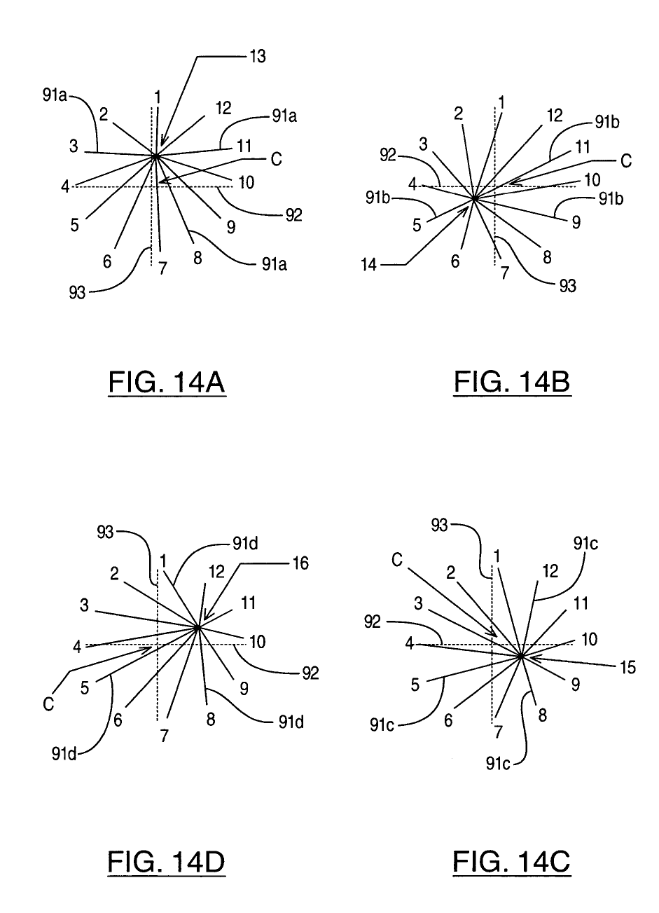

From FIGS. 11a, 11b, 11c, and 11d, it can be seen that four conical surfaces 60a, 60b, 60c, and 60d are required to describe all the impedance chords between the periareolar and base planes for when the electrode array 28 as illustrated in FIG. 2 is used on a breast, or other similarly shaped body part. It can be appreciated that different configurations of the electrode array and applications to different body parts can result in surfaces similar to 60a, 60b, 60c, and 60d, but having a different geometry. With electrode array 28, used for the preferred purpose of taking impedance measurements of the breast, then each of surfaces 60a, 60b, 60c, and 60d will contain twelve impedance chords, representing the connection of each periareolar plane electrode pair to twelve base plane electrode pairs, for a total of 48 impedance chords. For purposes of this application, these impedance chords are called conical plane impedance chords. FIGS. 14a, 14b, 14c, and 14d show projections (“shadows cast”) 91a, 91b, 91c, and 91d, respectively, of these conical plane impedance chords onto the body frontal plane.

In particular, FIG. 14a shows the projections 91a of the twelve impedance chords on the conical surface 60a from FIG. 11a onto the frontal body plane; FIG. 14b shows the projections 91b of the twelve impedance chords on the conical surface 60b from FIG. 11b onto the frontal body plane; FIG. 14c shows the projections 91c of the twelve impedance chords on the conical surface 60c from FIG. 11c onto the frontal body plane, and FIG. 14d shows the projections 91d of the twelve impedance chords on the conical surface 60d from FIG. 11d onto the frontal body plane. Lines 92 and 93 are orthogonal axes intersecting at point C, which represents the position of the nipple in the frontal body plane.

Co-pending application Ser. No. 09/749,613, which is incorporated herein by reference, describes a pixel plot method of data analysis for detecting the possible pr sence of a breast cancer. The breast electrode array that was subject of this application was circular in shape, and consisted of 16 equal length arms, each with an electrode pair close to the end of the arm. All impedance chords were, therefore, in the same plane (body frontal plane) and were represented as chords of a circle in the frontal plane. The circle was divided into equal size quadrants by orthogonal axes intersecting at the nipple. Briefly, pixel analysis consisted of subdividing the plane into a grid of square-shaped pixel elements, and calculating the impedance value of each pixel element from the number of impedance chords that pass through the pixel, the impedance magnitude of each such impedance chord, and the segment length of the chord within the pixel element. A pixel difference set was created by subtracting the pixel impedance values of homologous (mirror image) pixel elements in the right and left breasts. Analysis included calculating difference metrics from the means and sums of all of the difference values, and comparing to a pre-established difference threshold to diagnose the possibility of a disease state. Pixel difference sets can also be plotted (pixel plots) and be divided into sectors, with the sector displaying the largest difference being the likely location of a cancer for those sets where the calculated difference metric exceeds a threshold value.

The present invention generates three sets of pixel plots based on the method described above from application Ser. No. 09/749,613, one from each of the base, conical, and periareolar planes. However, as previously indicated, there are four separate conical surfaces, each defining impedance chords that can be projected onto the frontal plane, as shown in FIGS. 14a, 14b, 14c, and 14d. This would result in four impedance plots for conical impedance chords alone. It is therefore assumed, for the purpose of this invention, that an additive model can be used where the total effect of the conical surface impedance chords is the sum of their respective pixel plots. This can be done since each pixel plot has been mapped onto a common frame of reference, namely axes intersecting at the nipple.

It is also desirable to have a single, integrated pixel plot that combines base, conical, and periareolar pixel plots. This again would use an additive model where the base, conical, and periareolar plots are added. This single integrated pixel plot forms a fourth pixel plot.

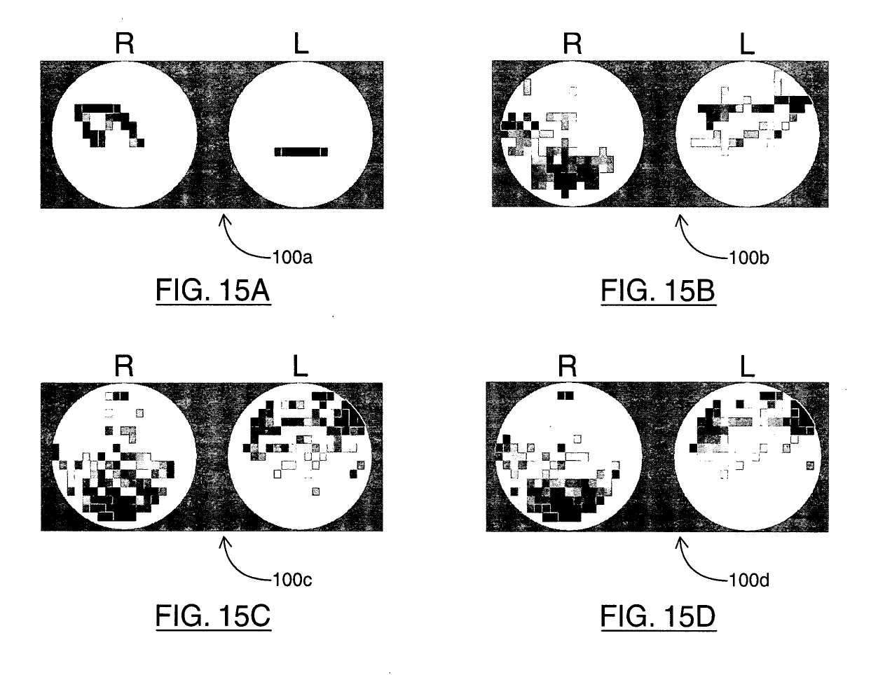

FIGS. 15a, 15b, 15c, and 15d are illustrative examples of pixel plots of this invention obtained from a normal subject. Pixel plots 100a. 100b, 100c, and 100d are periareolar, conical, base, and integrated pixel plots, respectively. Note that each consists of right (R) and left (L) breast pixel difference plots, with the magnitude of difference indicated here by a gray scale, with white or blank being no difference and black being maximum difference for a given plot. Following the convention of co-pending application Ser. No. 09/749,613, for any given pixel location, the value is plotted on the side having the lower value, or if there is no difference, the pixel area is left white or blank on both sides. Whereas the illustrated example of the present invention is a novel and improved apparatus and method for detecting and locating breast cancers, the invention can also be applied to other diseases or conditions in which there is a distinguishable difference in electrical impedance in the tissue as a result of the disease or condition.

It can be appreciated that variations to this invention would be readily apparent to those skilled in the art, and this invention is intended to include those alternatives.

We claim:

1. An electrode array for diagnosing the presence of a disease state in a living organism, the electrode array comprising:
a) a body;
b) a plurality of flexible arms extending from the body; and
c) a plurality of outer electrodes provided by the plurality of flexible arms, the outer electrodes arranged on the arms to obtain impedance measurements between respective electrodes and with at least one of the outer electrodes spaced from the body greater than the other outer electrodes.

2. An electrode array according to claim 1, wherein at least a further one of the outer electrodes is spaced from the body greater than the other outer electrodes but not as great as said at least one outer electrode.

3. An electrode array according to claim 2, wherein the further outer electrode is provided on a flexible arm adjacent to a flexible arm having the at least one outer electrode.

4. An electrode array according to claim 3, wherein the outer electrodes are arranged in electrode pairs, and each of the plurality of arms is provided with an electrode pair.

5. An electrode array according to claim 4, further comprising a plurality of inner electrodes provided on at least one of the flexible arms and positioned partway between the body and the outer electrodes.

6. An electrode array according to claim 5, wherein at least one of the inner electrodes is spaced from the body greater than the other inner electrodes.

7. An electrode array according to claim 6, wherein the at least one inner electrode is provided on the flexible arm having the at least one outer electrode.

8. An electrode array according to claim 7, wherein the inner electrodes are arranged in electrode pairs.

9. An electrode array according to claim 1, wherein the at least one outer electrode comprises a first set of electrodes having at least one electrode on each of two adjacent flexible arms.

10. An electrode array according to claim 9, wherein the outer electrodes provide for a second set of electrodes spaced from the body greater than the other outer electrodes but not as great as the first set of electrodes.

11. An electrode array according to claim 10, wherein the second set of electrodes has at least one electrode on each of two flexible arms, and said flexible arms are each adjacent to one of the flexible arms that has the first set of electrodes.

12. An electrode array according to claim 11, wherein the outer electrodes provide for a third set of electrodes spaced from the body greater than the other outer electrodes but not as great as the second set of electrodes.

13. An electrode array according to claim 12, wherein the third set of electrodes has at least one electrode provided on one flexible arm, and said flexible arm is adjacent to one of the flexible arms that has the second set of electrodes.

14. An electrode array according to claim 13, wherein the outer electrodes provide for a fourth set of electrodes spaced from the body greater than the other outer electrodes but not as great as the third set of electrodes.

15. An electrode array according to claim 14, wherein the fourth set of electrodes has at least one electrode on each of two flexible arms, and one of said flexible arms is adjacent the flexible arm that has the third set of electrodes, and the other of said flexible arms is adjacent one of the flexible arms that has the second set of electrodes.

16. An electrode array according to claim 15, wherein the remaining of the other outer electrodes are equally spaced from the body not as great as the fourth set of electrodes.

17. An electrode array according to claim 16, further comprising a plurality of inner electrodes provided on at least one of the flexible arms and positioned partway between the body and the outer electrodes.

18. An electrode array according to claim 17, wherein at least one of the inner electrodes is spaced from the body greater than the other inner electrodes.

19. An electrode array according to claim 18, further comprising a plurality of inner electrodes provided on at least one of the flexible arms and positioned partway between the body and the outer electrodes, and with at least one of the inner electrodes spaced from the body greater than the other inner electrodes and provided on one of the flexible arms having the first set of electrodes.

20. An electrode array according to claim 19, wherein at least one of the other inner electrodes is provided on one of the flexible arms having the second set of electrodes, but not adjacent to the flexible arm having the at least one inner electrode.

21. An electrode array according to claim 20, wherein at least one of the other inner electrodes is provided on the flexible arm having the third set of electrodes, and with this flexible arm not adjacent the flexible arm having both the second set of electrodes and said other inner electrodes.

22. An electrode array according to claim 21, wherein at least one of the other inner electrodes is provided on at least one other flexible arm that is not adjacent to any of the flexible arms that have the first, second, third, and fourth set of electrodes.

23. An electrode array according to claim 22, wherein the other inner electrodes are equally spaced from the body.

24. An electrode array according to claim 23, wherein the outer electrodes are arranged in electrode pairs, and each of the plurality of flexible arms is provided with an electrode pair.

25. An electrode array according to claim 24, wherein the inner electrodes are arranged in electrode pairs.

26. An electrode array according to claim 25, wherein the plurality of flexible arms are spaced around the body.

27. An electrode array according to claim 26, wherein the electrode array has twelve flexible arms spaced around the body.

28. An electrode array according to claim 27, wherein certain of the flexible arms are of different lengths to provide for the spacing of the different sets of electrodes.

29. An electrode array for diagnosing the presence of a disease state in a living organism, the electrode array comprising:
a) a body;
b) a plurality of flexible arms extending from the body;
c) a plurality of electrodes provided by the plurality of flexible arms, the electrodes arranged on the arms to obtain impedance measurements between respective electrodes; and
d) a marker provided along the central axis of at least one of the flexible arms.

30. An electrode array according to claim 29, wherein the marker is a line along the central axis of the flexible arm.

31. An electrode array according to claim 30, wherein at least the flexible arm with the marker is transparent.

32. An electrode array according to claim 31, wherein all the flexible arms are transparent.

33. An electrode array according to claim 32, wherein the flexible arm with the marker is provided with a tab at its end thereof.

34. A template for positioning an electrode array on a part of a living organism to be diagnosed for the presence of a disease state, the template comprising:
a) an elongate body; and
b) a mark provided over at least part of the length of the body, and wherein the elongate body has an opening therein and is provided with at least one hole spaced from the opening.

35. A template according to claim 34, wherein the elongate body has a central axis and the mark is on the central axis.

36. A template according to claim 35, wherein the mark is a line along the central axis.

37. A template according to claim 36, wherein the opening and the at least one hole are spaced from one another along the central axis.

38. A template according to claim 37, wherein the opening is sized to fit around a nipple of a breast.

39. A template according to claim 38, wherein the opening is provided at one end of the elongate body, and the elongate body is of sufficient length so that when the opening is fitted around the nipple of one breast the other end of the elongate body extends to at least the nipple of the other breast.

40. A template according to claim 39, wherein the mark extends to the other end of the elongate body.

41. A template according to claim 40, wherein the elongate body is transparent.

42. A system for positioning an electrode array on a part of a living organism to be diagnosed for the presence of a disease state, the system comprising:
a) a template having:
i. an elongate body; and
ii. a mark provided over at least part of the length of the body, and wherein the elongate body has an opening therein and is provided with at least one hole spaced from the opening; and
b) an electrode array having:
i. a body;
ii. a plurality of flexible arms extending from the body; and
iii. a marker provided along the central axis of at least one of the flexible arms.

43. A system according to claim 42, wherein the elongate body of the template has a central axis and the mark is on the central axis.

44. A system according to claim 43, wherein the mark is a line along the central axis.

45. A system according to claim 44, wherein the opening and the at least one hole are spaced from one another along the central axis.

46. A system according to claims 45, wherein the at least one hole in the elongate body of the template is three holes.

47. A system according to claim 46, wherein the opening in the elongate body of the template is sized to fit around a nipple of a breast.

48. A system according to claim 47, wherein the opening in the elongate body of the template is provided at one end thereof, and the elongate body is of sufficient length so that when the opening is fitted around the nipple of one breast the other end of the elongate body extends to at least the nipple of the other breast.

49. A system according to claim 48, wherein the mark extends to the other end of the elongate body.

50. A system according to claim 49, wherein the elongate body of the template is transparent.

51. A system according to claim 50, wherein the marker of the electrode array is a line along the central axis of the flexible arm.

52. A system according to claim 51, wherein at least the flexible arm of the electrode array with the marker is transparent.

53. A system according to claim 52, wherein all the flexible arms of the electrode array are transparent.

54. A system according to claim 53, wherein the flexible arm of the electrode array with the marker is provided with a tab at its end thereof.

55. A method of positioning an electrode array on a part of a living organism to be diagnosed for the presence of a disease state, the electrode array positioned using a template, the template having an elongate body and a mark provided over at least part of the length of the body, and wherein the elongate body has an opening therein and is provided with at least one hole spaced from the opening, the electrode array having a body a plurality of flexible arms extending from the body and a marker provided along the central axis of at least one of the flexible arms, the method comprising:
a) centering the opening in the template about a nipple of one breast;
b) positioning the template about the nipple until the mark on the template is at the center of the nipple of the other breast;
c) marking the living organism through the hole in the template;
d) removing the template and centering the electrode array about the nipple of the one breast; and
e) positioning the electrode array by aligning the marker provided on the at least one flexible arm to the marking on the living organism.

56. A method according to claim 55, wherein the elongate body of the template has a central axis and the mark is on the central axis.

57. A method according to claim 57, wherein the mark is a line along the central axis.

58. A method according to claim 57, wherein the opening and the at least one hole are spaced from one another along the central axis.

59. A method according to claim 58, wherein the at least one hole in the elongate body of the template is three holes to provide for markings on the living organism that represent different electrode array sizes.

60. A method according to claim 59, wherein the elongate body of the template is transparent.

61. A method according to claim 60, wherein the marker of the electrode array is a line along the central axis of the flexible arm.

62. A method according to claim 61, wherein at least the flexible arm of the electrode array with the marker is transparent.

63. A method according to claim 62, wherein all the flexible arms of the electrode array are transparent.

64. A method according to claim 63, wherein the flexible arm of the electrode array with the marker is provided with a tab at its end thereof.

65. A method of diagnosing the possibility of a disease state in one of first and second substantially similar parts of a living organism, the method comprising:
a) obtaining a plurality of impedance measurements taken between a predetermined plurality of points encircling a first area of the parts;
b) obtaining a plurality of impedance measurements taken between a predetermined plurality of points encircling a second area of the parts, the second area at a different topology on the part than the first area;
c) obtaining a plurality of impedance measurements taken from a predetermined plurality of points between the first area and the second area;
d) producing at least one pixel plot from a chord plot produced by the impedance measurements taken; and
e) analyzing the pixel plot to diagnose the possibility of a disease state.

66. A method according to claim 65, wherein the pixel plot is a first pixel plot derived from the impedance measurements taken from the first area.

67. A method according to claim 66, wherein the pixel plot is a second pixel plot derived from the impedance measurements taken from the second area.

68. A method according to claim 67, wherein the pixel plot is a third pixel plot derived from the impedance measurements taken from between the first area and the second area.

69. A method according to claim 68, wherein the third pixel plot is the sum of separate pixel plots that can be derived from the impedance measurements taken from between each point in the first area and the plurality of points in the second area.

70. A method according to claim 69, wherein the separate pixel plots that make the third pixel plot are all mapped onto a common frame of reference.

71. A method according to claim 70, wherein the separate pixel plots are all mapped onto a common reference plane.

72. A method according to claim 71, wherein the common frame of reference is a set of orthogonal axes intersecting a predetermined point of the part of the living organism to be diagnosed.

73. A method according to claim 65, wherein the pixel plot is a plurality of pixel plots comprising a first pixel plot derived from the impedance measurements taken from the first area, a second pixel plot derived from the impedance measurements taken from the second area of the breast, and a third pixel plot derived from the impedance measurements taken between the first area and the second area.

74. A method according to claim 73, wherein the third pixel plot is the sum of separate pixel plots that can be derived from the impedance measurements taken from between each point in the first area and the plurality of points in the second area.

75. A method according to claim 74, wherein the separate pixel plots that make the third pixel plot are all mapped onto a common frame of reference.

76. A method according to claim 75, wherein the common frame of reference is a set of orthogonal axes intersecting a predetermined point of the part of the living organism to be diagnosed.

77. A method according to claim 76, wherein the separate pixel plots are all mapped onto a common reference plane.

78. A method according to claim 77, wherein the common reference plane is the body frontal plane.

79. A method according to claim 78, wherein the first pixel plot, the second pixel plot, and the third pixel plot, are all mapped onto the common frame of reference.

80. A method according to claim 79, wherein the plurality of pixel plots further comprise an integrated plot combining the first pixel plot, the second pixel plot, and the third pixel plot.

81. A method according to claim 80, wherein the part of the living organism to be diagnosed is a breast.

82. A method according to claim 81, wherein the first area is the periareolar area of the breast and the first pixel plot is a periareolar pixel plot.

83. A method according to claims 82, wherein the second area is the base area of the breast and the second pixel plot is a base pixel plot.

84. A method according to claim 83, wherein the third pixel plot is a conical pixel plot derived from impedance measurements taken from a predetermined plurality of points between the periareolar area of the breast and the base area of the breast.

85. An electrode array for diagnosing the presence of a disease state in a living organism, the electrode array comprising:
a) a body;
b) a plurality of flexible arms extending from the body; and
c) a plurality of outer electrodes provided by the plurality of flexible arms; and
d) a plurality of inner electrodes provided on at least one of the flexible arms and positioned partway between the body and the outer electrodes,
and wherein the outer electrodes and the inner electrodes are arranged on the arms to obtain impedance measurements between respective electrodes.

86. An electrode array according to claim 85, wherein the outer electrodes are arranged in electrode pairs.

87. An electrode array according to claim 86, wherein the inner electrodes are arranged in electrode pairs.

88. An electrode array according to claim 87, wherein the electrode pairs comprise a current injection electrode and a voltage measurement electrode.

89. A system for diagnosing the possibility of disease in a body part, the system comprising:
a) electrode array containing a plurality of outer electrodes and at least one inner electrode capable of being electrically coupled to the body part;
b) a controller switching unit; and
c) a multiplexing unit,
and wherein the controller switching unit and multiplexing unit allow a current to flow between any two electrodes and a resultant voltage measurement to be measured between any two electrodes.

90. The system according to claim 89, wherein the outer electrodes are arranged in electrode pairs.

91. The system according to claim 90, wherein the inner electrodes are arranged in electrode pairs.

92. The system according to claim 91, wherein the controller-switching unit and the multiplexing unit allows any one of the inner electrodes and outer electrodes to be a current injection electrode, and allows any one the inner electrodes and outer electrodes to be a voltage measurement electrode.

93. The system according to claim 92, wherein the controller-switching unit and the multiplexing unit select the current injection electrodes and the voltage measurement electrodes such that a tetrapolar measurement is taken between any two pairs of inner electrodes, any two pairs of outer electrodes, and any two pairs of electrodes with one selected from the pairs of outer electrodes and one selected from the pairs of inner electrodes.

94. Use of an electrode array for diagnosing the presence of a disease state in a living organism, the electrode array comprising a body, a plurality of flexible arms extending from the body, a plurality of outer electrodes provided by the plurality of flexible arms, and a plurality of inner electrodes provided on at least one of the flexible arms and positioned partway between the body and the outer electrodes, the outer electrodes and the inner electrodes are arranged on the arms to obtain impedance measurements between respective electrodes, and wherein the impedance values are arranged in a mathematical matrix and mathematical analysis is performed to diagnose for the presence of a disease state.
